(19) United States
(12) Patent Application Publication
(10) Pub. No.: US 2024/0068981 A1
(43) Pub. Date: Feb. 29, 2024

(54) Biosensor Based Tool to Monitor Obesity

(71) Applicant: Board of Regents, The University of Texas System, Austin, TX (US)

(72) Inventors: Md Nurunnabi, El Paso, TX (US); Juan C. Noveron, El Paso, TX (US); Md Ariful Ahsan, El Paso, TX (US); Tamanna Islam, El Paso, TX (US)

(21) Appl. No.: 18/364,425

(22) Filed: Aug. 2, 2023

Related U.S. Application Data
(60) Provisional application No. 63/370,147, filed on Aug. 2, 2022.

(51) Int. Cl. G01N 27/327; G01N 27/36

(52) U.S. Cl. CPC G01N 27/3275; G01N 27/36; G01N 2333/575

(57) ABSTRACT

A leptin detecting device is provided. The device comprises a counter electrode, a reference electrode, and a working electrode having an electroactive surface area. A Fe:Ni/C layer deposited is deposited on the counter electrode, reference electrode, and working electrode, wherein deposit of a biological tissue sample containing a concentration of leptin above a threshold level onto the Fe:Ni/C layer produces an electric current through the counter electrode, reference electrode, and working electrode. A circuit is connected to the counter electrode, reference electrode, and working electrode, wherein the circuit is configured to produce a specified waveform in response to the electric current produced by the presence of leptin above the threshold level.

### CROSS-REFERENCE TO RELATED APPLICATION

This application claims the benefit of U.S. Provisional Patent Application Ser. No. 63/370,147, filed Aug. 2, 2022, and entitled “Biosensor Based Tool to Monitor Obesity,” which is incorporated herein by reference in its entirety.

### BACKGROUND INFORMATION

### 1. Field

The present disclosure relates to biosensors, and more specifically to a biosensor device for detecting circulating leptin levels.

### 2. Background

The world has seen an exponential rise of severe obesity in both adults and children in the recent years, making it a public health crisis. The recent Behavioral Risk Factor Surveillance System (BRFSS) data showed that the adult obesity rate has increased by 35% in almost 16 states in the United States. Obesity is one the key reasons for metabolic abnormalities in the lipid and glucose. Thereby, obesity is very closely linked with diabetes (types 1 and 2) and cardiovascular diseases. Obesity is also responsible for various cancers and arthritis. Moreover, recent studies have shown that obese COVID-19 patients are more likely have severe conditions and higher death count. Therefore, it is imperative to identify obesity markers at an earlier stage in order to prevent obesity related complications and diseases.

Therefore, it would be desirable to have a method and apparatus that take into account at least some of the issues discussed above, as well as other possible issues.

### SUMMARY

An illustrative embodiment provides a leptin detecting device. The device comprises a counter electrode, a reference electrode, and a working electrode having an electroactive surface area. A Fe:Ni/C layer deposited is deposited on the counter electrode, reference electrode, and working electrode, wherein deposit of a biological tissue sample containing a concentration of leptin above a threshold level onto the Fe:Ni/C layer produces an electric current through the counter electrode, reference electrode, and working electrode. A circuit is connected to the counter electrode, reference electrode, and working electrode, wherein the circuit is configured to produce a specified waveform in response to the electric current produced by the presence of leptin above the threshold level.

Another illustrative embodiment provides a biosensor comprising a housing and a removable sensor electrode inserted into the housing. The sensor electrode comprises a Fe:Ni/C layer, wherein deposit of a biological tissue sample containing a concentration of leptin above a threshold level onto the Fe:Ni/C layer produces an electric current through the sensor electrode. A circuit inside the housing is electrically connected to the sensor electrode, wherein the circuit is configured to produce a specified waveform in response to the electric current produced by the presence of leptin above the threshold level.

Another illustrative embodiment provides a leptin biosensor comprising a handheld housing and a screen printed carbon electrode (SPCE) removably inserted into the handheld housing. The SPCE comprises a counter electrode, a reference electrode, and a working electrode. A Fe:Ni/C layer is deposited on the SPCE, wherein deposit of a blood sample on the Fe:Ni/C layer produces an electric current through the SPCE if the blood sample contains a concentration of leptin above a threshold level. A circuit is electrically connected to the SPCE, wherein the circuit is configured to produce a specified waveform in response to the electric current produced by the presence of leptin above the threshold level. A display is the housing displays a readout of detected lepton levels in response to the waveform produced by the circuit.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 depicts a diagram illustrating a process for synthesizing FeNi/C nanohybrid in accordance with an illustrative embodiment;

FIG. 2 depicts a diagram of an electrode setup in accordance with an illustrative embodiment;

FIG. 3 depicts a diagram showing a step-by-step process for modifying the electrode for Lep detection in accordance with an illustrative embodiment;

FIG. 4 depicts a diagram illustrating a process for sensor modification for SPR analysis in accordance with an illustrative embodiment;

FIG. 5 depicts a diagram illustrating sample collection and electrochemical detection using GCE in accordance with an illustrative embodiment;

FIG. 6 depicts a diagram illustrating sample collection and electrochemical detection using SPCE in accordance with an illustrative embodiment;

FIG. 7 depicts a diagram illustrating a leptin sensitive electrode in accordance with an illustrative embodiment;

FIG. 8 depicts a diagram illustrating a biosensor device configured to detect leptin in accordance with an illustrative embodiment;

FIG. 9 depicts a circuit diagram of a leptin level processing circuit in accordance with an illustrative embodiment; and

FIG. 10 depicts a block diagram illustrating a leptin detecting biosensor device in accordance with an illustrative embodiment.

### DETAILED DESCRIPTION

The illustrative embodiments recognize and take into account one or more different considerations. For example, the illustrative embodiments recognize and take into account that the world has seen an exponential rise of severe obesity in both adults and children in the recent years, making it a public health crisis. The recent Behavioral Risk Factor Surveillance System (BRFSS) data showed that the adult obesity rate has increased by 35% in almost 16 states in the US.

The illustrative embodiments also recognize and take into account that obesity is one the key reasons for metabolic abnormalities in the lipid and glucose. Thereby, obesity is very closely linked with diabetes (types 1 and 2) and cardiovascular diseases. Obesity also responsible for various cancer and arthritis.

The illustrative embodiments also recognize and take into account that leptin (Lep) is an adipose tissue secreted 16-kDa protein hormone synthesized by ob gene that is linked with regulating mammalian appetite through the body's energy status. Lep controls the nutritional needs of the body by interacting with the hypothalamus. Lep concentration in humans varies with sex, age, and genetic abnormality of the individual.

The illustrative embodiments also recognize and take into account that the normal blood concentrations of Lep in males and females are ˜11.1 and ˜5.6 ng/mL, respectively. However, those with obese conditions can have Lep concentration of ˜100 ng/mL, and Lep gene abnormalities can result in 300-700 ng/mL concentration. A slight increase of Lep would usually lead to the reduction of the appetite and hence, helps maintaining the body mass. However, in obese conditions, although adipose tissue produces copious amounts of Lep in the body, the potentiality of Lep to show anorexic effect on the brain is reduced. This situation is termed “Leptin resistance,” wherein Lep signaling in the hypothalamus region of the brain gets disrupted due to the blockage of intracellular signaling associated with impaired Lep receptor or lowering of Lep transportation through the blood brain barrier (BBB). Because of its huge significance in obesity progression, Lep resistance is now believed to be one of the key conditions for diagnosing obesity.

The illustrative embodiments also recognize and take into account that, as Lep is an important biomarker of obesity, it is imperative to develop highly effective sensors that will be able to detect Lep in the physiological concentration from real samples with accuracy.

The illustrative embodiments also recognize and take into account that the most widely used methods for Lep detection include: radioimmunoassay, SPR, enzyme-linked immunoassay (ELISA), capillary electrophoresis, and electrochemical biosensors (ECBs). High sensitivity, good anti-interference ability, cost-effective, minimal sample preparation, and ease of operation are the key benefits of ECBs. Among different kinds of ECBs, the immunosensors are advantageous as they can selectively detect specific analytes through bio-affinity.

The illustrative embodiments also recognize and take into account that one of the key areas of improvement for immunosensors is the development of highly electroactive and biocompatible catalytic materials. Lep has a quaternary structure with an active disulfide bond. This structure of the active site can be exploited for highly efficient detection of Lep. Due to clinical potential, various metallic, graphene, porous carbon, and polymer based catalytic systems have been utilized for enhancing the sensitivity of the immunosensors for Lep detection.

The illustrative embodiments also recognize and take into account that metallic nanoparticles embedded within porous carbon structures have shown good electrocatalytic activity in diverse areas of electrochemical research. Those with hetero-metallic systems are more promising as electrocatalysts compared to their mono-metallic counterparts. The cations of the hetero-metallic systems are chosen to facilitate compatibility of the metal ions with each other. The metallic π-π interactions lead to synergetic increase in their electrocatalytic activity, while the carbon base provides support that stabilizes the metallic nanoparticle.

The illustrative embodiments provide an impedimetric sensor for diagnosing leptin resistance based on three hetero-metallic FeNi/C systems having different metallic ratios with excellent biocompatibility and conductivity. The morphological properties, degree of defects, chemical composition, functional groups and electrochemical, properties of the materials were investigated and the suitability of each of the three electrocatalysts for biomolecules immobilization and subsequent sensing feasibility were assessed. As per analysis, Fe:Ni(1:1)/C system provided superior efficacy for anti-Lep immobilization and further immuno-conjugation.

The binding affinity of anti-Lep with the electrocatalyst was further confirmed through surface plasmon resonance (SPR) sensorgram analysis. Upon determination of the most effective material for Lep detection, we utilized the device for the detection of Lep over a concentration window using both glassy carbon electrodes (GCEs) and screen printed carbon electrodes (SPCEs). After laboratory data acquisition, the sensor was implemented for serum analysis collected from a set of obese rats, induced by high fat diet, which demonstrated excellent outputs comparable with the laboratory analysis. The stability, performance, reproducibility, and selectivity of the Device were further studied to demonstrate the adequacy and potency. The device was found very sensitive as it can detect 157.4, and 184.9 fg/mL, with GCE and SPCE, respectively. With further validation and optimization utilizing patient derived serum samples the device has strong potential for clinical translation for real-time diagnosis of Lep resistance obesity.

Iron nitrate (Fe(NO₃)₃.6H₂O) and nickel nitrate (Ni(NO₃)₂·6H₂O) were purchased from Sigma Aldrich. Deionized water (DIW) was used from a Milli-Q® instrument (Millipore Corporation). Tissue paper was purchased from a local store. Biomolecules used in this work such as recombinant human Lep protein, anti-Lep (polyclonal, pAb), interleukin-6 (IL 6), troponin I₃ (TnI₃), and vascular cell adhesion protein1 (VCAM1) were purchased from ABclonal Science Inc (Woburn, MA). Bovine serum albumin (BSA), potassium ferricyanide (K₃Fe(CN)₆), mono and dibasic potassium phosphate (KH₂PO₄ and K₂HPO₄), potassium chloride (KCl), and glucose were purchased from Thermo Fisher Scientific Inc. N-(3-Dimethylaminopropyl)-N-ethylcarbodiimide hydrochloride (EDC·HCl), and N-hydroxysuccinimide (NHS) were purchased from sigma Aldrich, USA. All experiments were carried out using distilled water (electrolytes concentration −1.3 ppb). For electrochemical sample preparation, all biomolecules' solutions were prepared in 0.1 M phosphate buffer saline (PBS) solution. A 5 mM concentrated K₃Fe(CN)₆ solution was used in all electrochemical experiments and was prepared by dissolving it in 1.0 M KCl solution. The PBS solution of concentration 0.1 M was prepared by mixing KH₂PO₄ and K₂HPO₄ in distilled water.

Initially, 1.5 g of each metal salts were dissolved in 10 mL of DIW in a 100 mL beaker. Subsequently, 3.0 g of facial tissue (in the form of tiny pieces) was placed into the metal salt solution and the bi-mixture was sonicated so that the Fe³⁺ and Ni²⁺ ions were adsorbed on the facial tissue, followed by heating the mixture at 100° C. to remove all the water molecules. Afterward, the mixture was carbonized at 800° C. for 4 hr in a tube furnace and the heating rate was set to 5° C. min-1. The black product of bimetallic nanoparticles was collected and named as Fe:Ni(1:1)/C. Another two other bimetallic electrocatalysts, namely Fe:Ni(3:1)/C and Fe:Ni(1:3)/C were prepared with respect to different weight ratios of the iron and nickel salts that were used during the synthesis process.

The electrochemical sensor was constructed by following a layer-by-layer modification process. At first the GCE was polished with a 0.05 μm alumina slurry for 30 min to remove any adsorbed oxide layers. Then the electrode was washed three times with distilled water and dried in room temperature. A value of 3 μL of the nanomaterial suspension (in methanol, 5 mg/mL) was drop-casted on the electrode surface and dried in air. Once the electrode surface was dried, a mixture of 10 mg/mL EDC·HCl and 6 mg/mL NHS was immobilized on the electrode surface by incubating in the EDC-NHS solution for 30 min. The EDC/NHS solution is a well-known cross linker which is able to initiate an active end that can efficiently bind with the —NH₂ group of the antibody by creating —CO—NH-linkage.

The EDC-NHS/FeNi/C_GCE electrode was then immobilized in the anti-Lep solution to prepare an antibody modified electrode. To block any nonspecific binding sites on the electrode surface, the modified electrode was incubated in a 0.5% BSA solution. The final electrode was termed as BSA/anti-Lep/EDC-NHS/FeNi/C GCE and stored at 4° C. until use. Before conducting electrochemical experiment, the electrode was incubated in the Lep solution and dried at 4° C. The same procedure was further followed for constructing a SPCE based Lep biosensor.

The electrode incubation times in different solutions, especially, in the anti-Lep, BSA and Lep solutions, were optimized by observing the changes in the EIS signals after incubating electrodes in different time periods (30, 45, 60, and 90 mins). It is worth noting that the electrode was kept in 4° C. while incubating in different biomolecular solutions for different time periods and washed carefully with distilled water after each modification to ensure complete removal of any substances that were not bound to the electrode. All biorecognition molecules were diluted in 0.1 M PBS solution.

The bimetallic FeNi nanoparticles were first characterized using X-ray diffraction (XRD) spectroscopy with a Cu-Kα radiation source in 2θ=20-80° at a scan rate of 5° C. min⁻¹ (Model: Bruker D8 advanced diffractometer). Transmission electron microscopic (TEM) analysis was carried out on a Hitachi H-7650 microscope to further inspect the morphology of the nanoparticles. The surface elements and chemical valence of the nanoparticles were detected using X-ray photoelectron spectroscopy (XPS) on a PHI VersaProbe II Scanning XPS Microprobe with scanning monochromatic X-ray Al Kα radiation as the excitation source (200 μm area analyzed, 52.8 W, 15 kV, 1486.6 eV) and a charge neutralizer.

For biomolecule-electrocatalyst binding affinity study, an iMSPR instrument was used (iCLUEBiO Co. Ltd, Republic of Korea). The Au-chips used for the SPR analysis were also purchased from iCLUEBiO Co. Ltd. All electrochemical experiments, namely, electrochemical impedance spectroscopy (EIS) and cyclic voltammetry (CV) were performed using an CHI660E electrochemical workstation, CHI Inc, USA. A three electrodes setup was utilized where both classical GCE and SPCE were used as the working electrode, Ag/AgCl as the reference, and Pt wire as the counter electrode and were purchased from CHI Inc, USA.

Six Sprague-Dawley rats (male/female) were chosen for preparing obese rat model. The rats were fed high fat diet (HFD, extra 60% calories) for 15 days to induce obesity through observing the changes in their body weight every day. For comparison, another group of six rats were considered which were given normal fat diet (NFD, 10% calories) every day. After creating the obese rat model, both NFD and HFD rats were sacrificed, and the blood was collected. The blood was centrifuged at 1500 rpm for 10 mins to separate the serum. For electrochemical measurements, the serum samples of the NFD rats were first diluted 50 times in normal PBS and spiked with two different concentrations of Lep (500 pg/mL and 1 ng/mL), while the serum of the HFD rats were directly used without any spiking procedures.

For constructing the modified electrodes, the same layer-by-layer modification process was followed. The final modified electrode was incubated in diluted spiked serum (NFD)/whole serum (HFD) for Lep quantification.

For electrochemical characterization of the FeNi bimetallic system and Lep signaling, both CV and EIS techniques were utilized. To evaluate the conjugation of the biomolecules with the sensor, the changes in the redox peak responses of potassium ferricyanide at the electrode-electrolyte interface were investigated which was achieved by taking the solution of K₃Fe(CN)₆ in the electrochemical cell. The frequency range used for EIS analysis was 1 Hz (low) to 1 MHz (high), and the initial potential was chosen based on the formal potential of potassium ferricyanides obtained by averaging the oxidation and reduction peak potentials from the CV experiment.

$${E}_{f} = \frac{{E}_{pa} + {E}_{pc}}{2} \qquad ( 1 )$$

To evaluate the applicability of the proposed sensor, we performed a quantitative analysis of Lep by using EIS technique over a wide concentration range. The value of charge transfer resistance (R_{c}t) was determined for each of the EIS data and plotted against the concentration of Lep (calibration plot). By using the intercept and slope of the calibration plot, the LOD, sensitivity and linear range of the sensor for Lep were determined.

$$LOD = \frac{3.3 \times SE}{s} \qquad ( 2 )$$

where, SE refers to the standard deviation and s as the slope of the calibration plot.

The selectivity, sensitivity, and specificity of the sensor were evaluated by performing interference analysis. A variety of biomolecules that are expected to present in the serum sample were used in which the modified electrode was incubated to see whether the sensor shows any affinity towards those biomolecules. The percentages of R_{ct} obtained for incubating the modified electrodes in the interfering agents were compared with the value of R_{ct} obtained for Lep only. While on the other hand, for evaluating the sensitivity and the specificity of the sensor, a mixture of Lep and the interfering agents was used where the modified electrode was incubated.

Once the sensor selectively bound with Lep from the mixture, the EIS experiment was carried out and the standard deviation in the obtained EIS signal was evaluated with respect to the response obtained for Lep only to determine the sensitivity and the specificity of the sensor. To test the sensor's applicability in detecting Lep from a real sample, the serum of the NFD/HFD rats was separated and prepared as described above. For assessing the performance of the sensor, the signal stability, storage stability, and the reproducibility of the EIS signal regardless of the number of electrodes were investigated. For these purposes, consecutive EIS analysis, signal acquisition of the modified electrode after more than a week and EIS of six different GCE electrodes were performed.

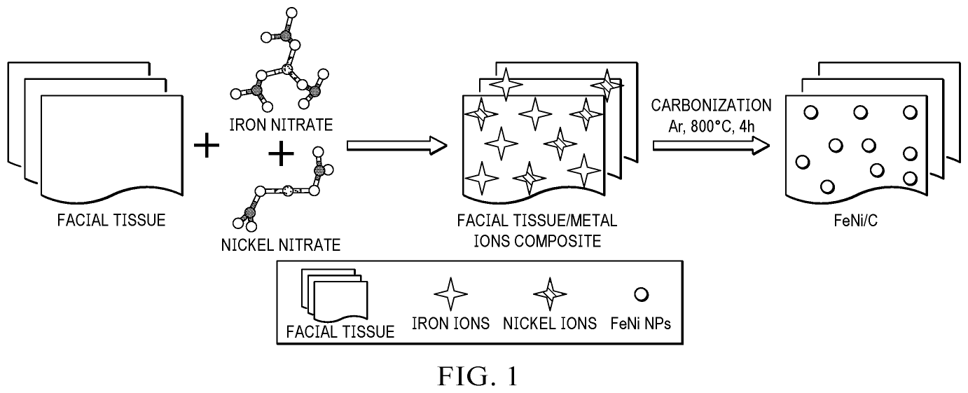

FIG. 1 depicts a diagram illustrating a process for synthesizing FeNi/C nanohybrid in accordance with an illustrative embodiment. As shown in FIG. 1, the synthesis of FeNi/C nanocomposite electrocatalyst is accomplished via a two-step strategy that includes the adsorption of the Ni²⁺/Fe³⁺ ions into facial tissue followed by pyrolysis. During the pyrolysis step, the facial tissue serves as a carbon source, while the metal salts act as a metal source to form the final carbon encapsulated FeNi structure.

The crystalline structure of the bimetallic NPs was first investigated using XRD. The XRD pattern showed three characteristic diffraction peaks in all cases at ˜44.22°, 51.25°, and 74.89°, corresponding to the 111, 200, and 220 crystal planes of the cubic FeNi alloy (JCPDS no. 65-3244), confirming the successful formation of the FeNi nanocomposite. However, the diffraction pattern did not show any additional peaks, suggesting the high purity of the as-prepared FeNi alloy.

Raman spectroscopic analysis was further performed for investigating the structural distinction of as-prepared FeNi/C nano electrocatalysts. There are mainly two typical peaks, that can be distinctly detected at ˜1339 cm⁻¹ and 1590 cm⁻¹ for all of the samples, assigned to the D and G band, respectively. The D band is associated with a breathing mode of K-point photons of A_{1g} symmetry, while G band arises from the first order scattering of the E_{2g} phonon of sp²-banded carbon atoms. The intensity ratio of these two bands (I_{D}/I_{G}) is sensitive to the disorder degree, and its increasing value manifests the formation of defects. The I_{D}/I_{G} values of the as-prepared three nanohybrids are higher than one, indicating that higher degree of defects is introduced into the composites during the bimetallic nano electrocatalyst's formation, which is beneficial to improving the electrocatalytic properties.

XPS analysis was further performed to examine the valence states and chemical composition of the FeNi/C nanocomposite. The survey scan of the XPS spectra showed the presence of carbon, oxygen, iron and nickel elements in the Fe:Ni(1:1)/C nano electrocatalyst sample. The high resolution C1s spectra was deconvoluted into four bands that corresponds to the C—O—C/C—OH, C—C/C—H, C═O and O—C═O.

The O is peak was deconvoluted into four peaks that correspond to the Ni—O/Fe—O, C—O, C═O and —OH. The existence of the oxygenated functional groups might provide more nucleation sites to favor the growth of the bimetallic nanocomposites.

Ni 2p band primarily comprises Ni 2p_{1/2} and Ni 2p_{3/2}, each of which was then fitted into two peaks corresponding to the Ni⁰ and Ni²⁺. Ni²⁺ peak usually corresponds to the nickel oxide and this could happen because of the air exposure, which might favor the formation of a thin layer of metal oxide as the Ni NPs are air sensitive.

Likewise, Fe 2p band is also mainly consisting of Fe 2p_{1/2} and Fe 2p_{3/2}. They were fitted into three peaks that corresponds to Fe⁰, Fe²⁺ and Fe³⁺. Therefore, the presence of metallic iron and nickel in the FeNi/C demonstrates the successful synthesis of FeNi bimetallic nanocomposites.

The morphology of the nanocomposite was then inspected using transmission electron microscopic analysis. The FeNi bimetallic NPs are uniformly dispersed and anchored into the porous carbon framework in the case of all of the FeNi bimetallic nanocomposites. Hence, the porous carbon framework plays a dual role being a substrate and protecting layer for the bimetallic NPs and protecting them from further oxidation. The average particle size of the nanocomposite was found to be ˜15-20 nm in diameter. Furthermore, the energy dispersive X ray spectroscopy (EDS) elemental color mapping experiments of Fe:Ni(1:1)/C nanocomposite showed the presence of all expected elements such as carbon, oxygen, nickel and iron.

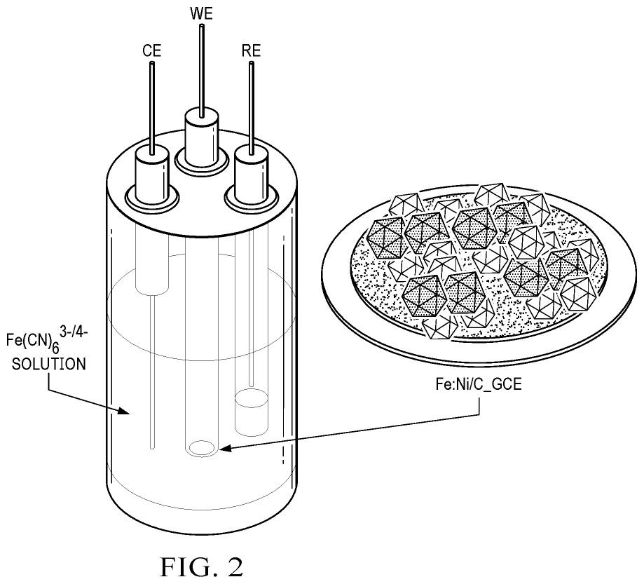

FIG. 2 depicts a diagram of an electrode setup in accordance with an illustrative embodiment. The electrochemical properties of FeNi/C nanocomposites were analyzed using standard 5 mM K₃[Fe(CN)₆] in 1.0 M KCl solution. The CV plots for polished GCE, Fe:Ni(1:1)/C, Fe:Ni(1:3)/C, and Fe:Ni(3:1)/C NPs show distinguishing features for the different FeNi/C nanocomposites compared to polished GCE. The GCE showed redox peaks for [Fe(CN)₆]³⁻↔[Fe(CN)₆]⁴⁻ around 0.24 and 0.33 V (ΔEₚ=90 mV). The peak current densities (J) for the reduction and oxidation processes were determined to be −0.62 and 0.59 mA/cm², respectively. The 0.96 ratio of Jₚₐ/J_{pc} and 90 mV ΔEₚ shows that [Fe(CN)₆]^{3-/4-} redox process followed a reversible reaction pathway on the GCE.

Once modified with the FeNi/C nanocomposites the CV shape drastically changed, clearly indicating the surface modification of GCE. The Fe:Ni(1:1)/C_GCE, Fe:Ni(1:3)/C_GCE, and Fe:Ni(3:1)/C_GCE showed multiple redox peaks in their CV response. In the case of Fe:Ni(1:1)/C_GCE the redox pair for [Fe(CN)₆]^{3-/4-} is observed around 0.23 (J_{pc}=−0.81 mA/cm²) and 0.33 V (Jₚₐ=0.79 mA/cm²). The Fe:Ni(1:3)/C_GCE had the redox peaks around 0.22 (J_{pc}=−0.7 mA/cm²) and 0.35 V (Jₚₐ=0.67 mA/cm²), while the Fe:Ni(3:1)/C_GCE showed the redox peaks around 0.25 (J_{pc}=−0.42 mA/cm²) and 0.32 V (Jₚₐ=0.48 mA/cm²). The ΔEₚ and Jₚₐ/J_{pc} ratios for all three FeNi/C electrode systems are close to those for reversible redox systems. However, the Fe:Ni(1:1)/C_GCE had the highest J values, indicating that the Fe:Ni(1:1)/C nanocomposite system was most active for the K₃[Fe(CN)₆] redox process.

All three modified electrodes showed a weak signal for another redox pair around 0.5 V. This peak was for the Fe^{2+/1+} oxidation states of the FeNi/C composite. The CV of Fe:Ni(3:1)/C_CGE showed the highest J around 0.5 V. The peak pair around 0.65 V is likely for the Ni^{1-/2+} of the FeNi nanocomposites. The peak increased as the Ni ratio is increased in the Fe:Ni(1:3)/C nanocomposite. Aside from these the Fe:Ni(3:1)/C showed another peak around 0.13 V. This peak is likely observed for the Fe^{0/1+} oxidation process. However, this peak is very unstable and decreases with each CV and ultimately disappears after the third consecutive CV. After that the CV shows steady response. These CVs indicated that the high iron content of Fe:Ni(3:1)/C material is less stable compared to the other two. The consecutive CV analysis (50 CVs) also confirmed the stability of the Fe:Ni(1:1)/C over two other ratios indicating its efficiency for biomolecule conjugation and immobilization. The scan rate variation data also shows that the K₃[Fe(CN)₆] redox process follows diffusion controlled process for all three materials.

All of these electrode systems could be described using the modified Randle's circuit. The charge transfer resistance (R_{ct}) for the GCE, Fe:Ni(1:1)/C_GCE, Fe:Ni(1:3)/C_GCE, and Fe:Ni(3:1)/C_GCE were determined to be 245.1, 445.6, 324.2, and 223.4Ω, respectively. These values indicate that Fe:Ni(3:1)/C material has the lowest resistance to charge transfer for the K₃[Fe(CN)₆] while, Fe:Ni(1:1)/C material has the highest resistance. But the CV response showed the highest J value for the Fe:Ni(1:1)/C_GCE. The reason can be explained based on the charging current distribution. Compared to the two other nanocomposites, Fe:Ni(1:1)/C shows relatively low density of peaks other than [Fe(CN)₆]^{3-/4-} redox pair and hence low charging current which could probably be responsible for the increment of R_{ct} for the Fe:Ni(1:1)/C nanocomposite. Also, the XPS data showed that, for the Fe:Ni/C nanocomposites the partial negative charge containing groups (—OH^{δ-}, O^{δ-}, O—C^{δ-}, etc.), these are very likely to repel the high negative charge bearing [Fe(CN)₆]^{3-/4-}, resulting in higher resistance to charge transfer. The EDS data also indicated high negative functional group content on Fe:Ni(1:1)/C material. This high surface area would allow for more [Fe(CN)₆]^{3-/4-} to interact with the Fe and Ni cations of Fe:Ni(1:1)/C composite. The presence of functional groups and high surface area indicates that the Fe:Ni(1:1)/C could have good bonding with the immobilized antibodies for better Lep sensitivity.

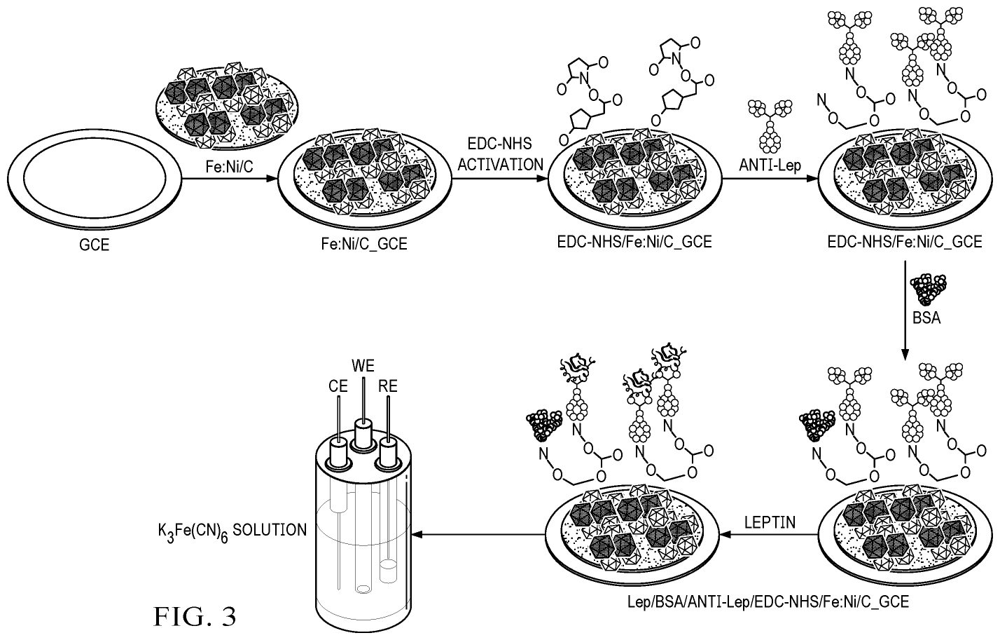

FIG. 3 depicts a diagram showing a step-by-step process for modifying the electrode for Lep detection in accordance with an illustrative embodiment. CV and EIS techniques were used for determining the Lep sensing capabilities of the Fe:Ni(1:1)/C, Fe:Ni(1:3)/C, and Fe:Ni(3:1)/C materials. The CV response increased after the initial modification with the Fe:Ni(1:1)/C material. In the next step EDC-NHS was immobilized on the Fe:Ni(1:1)/C_GCE through incubation. During this step, the current signal decreased. The reason for this could be the surface crowding and repulsion of the K₃[Fe(CN)₆] by the negative charge bearing functional groups of EDC-NHS. On the next step the EDC-NHS/Fe:Ni(1:1)/C_GCE was modified with anti-Lep. The anti-Lep modification showed further change in the CV signal. In the last step BSA was added to minimize nonspecific binding. The BSA/anti-Lep/EDC-NHS/Fe:Ni(1:1)/C_GCE showed the lowest current signal, as expected. The Fe:Ni(1:3)/C and Fe:Ni(3:1)/C materials were modified following the similar protocols.

The electrode modification steps were also analyzed with CV and EIS techniques. After each modification of the electrode the impedance signal showed clear changes, indicating that the electrode surface underwent modification. The highest impedance was observed for BSA/anti-Lep/EDC-NHS/Fe:Ni/C_GCE.

For 500 pg/mL of Lep in 5 mM K₃[Fe(CN)₆] solution, each of the FeNi/C materials showed distinct responses for the same concentration of Lep solution. The CV data showed that the Lep/BSA/anti-Lep/EDC-NHS/Fe:Ni(1:1)/C_GCE electrode had the smallest current response for the 500 pg/mL Lep solution. EIS results for the Lep/BSA/anti-Lep/EDC-NHS/FeNi/C_GCE electrodes show that the Fe:Ni(1:1)/C nanocomposite had the largest EIS signal for the Lep. From both CV and EIS it was clear that Fe:Ni(1:1)/C material is the best candidate for Lep detection.

Optimization of the incubation time and concentration of anti-Lep showed that 100 ng/mL of antibody is best suitable and 60 minutes is optimized time period for anti-Lep immobilization, while for BSA, 90 minutes is the most suitable time period for BSA immobilization. The anti-Lep binding capability of the Fe:Ni(1:1)/C material was confirmed through SPR analysis.

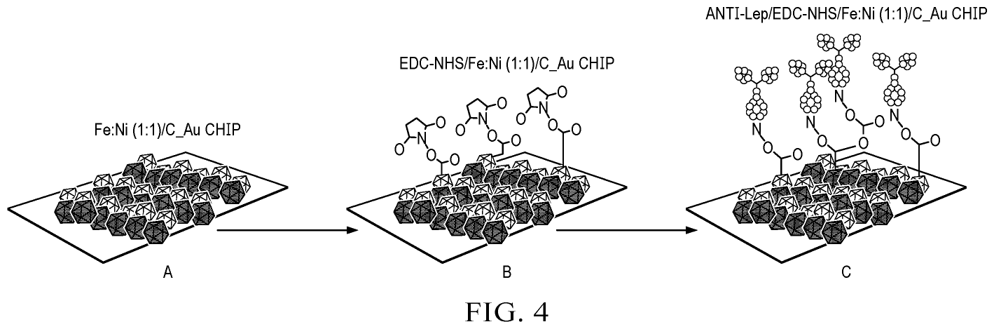

FIG. 4 depicts a diagram illustrating a process for sensor modification for SPR analysis in accordance with an illustrative embodiment. Initially the Fe:Ni(1:1)/C nanocomposite was dispersed in the EDC-NHS solution and was immobilized on the Au-chip for preparing the nanocomposite modified Au chip. Next, the EDC-NHS solution was passed over the sensor using the flow injector. The SPR sensorgram showed a good response around 150s when the Fe:Ni(1:1)/C_Au sensor interacted with the —COOH group of EDC-NHS. Finally, the anti-Lep solution was injected on the EDC-NHS/Fe:Ni(1:1)/C_Au chip, which showed another change in the SPR sensorgram around 500s. This change in the sensorgram was due to the binding of the —NH₂ group of anti-Lep with the EDC-NHS. The sensorgram showed a declining trend as over time the anti-Lep dissociated from the EDC-NHS/Fe:Ni(1:1)/C_Au sensor surface. However, the sensorgram showed minimal change in its response over a time period of 500s. This response clearly indicated that the anti-Lep and EDC-NHS/Fe:Ni(1:1)/C interaction was steady.

The performance of the biosensor for quantitative measurement of the Lep is studied using EIS technique. In general, the normal Lep level in human plasma sample is approximately ˜20 ng/mL. In obese humans, the level of the Lep can reach up to ˜100 ng/mL, while the content in human adipose tissue under obese conditions varies from 17.6 to 20 ng/mL. The proposed sensor was tested to see if it could accurately sense Lep hormone and what is the limit of detection (LOD) of the sensor for Lep.

EIS plots of the modified sensor obtained for several concentrations of Lep ranging from 500 fg/mL up to 100 ng/mL indicate that the R_{c}t of each of the electrodes increases proportionally with increasing the level of Lep in solution up to 80 ng/mL. A linear relationship exists between the obtained R_{c}t with Lep concentration indicating that the sensor has a large concentration range for Lep. The R² value indicates a better fitting for the calibration plot. The value of LOD is calculated using the slope of the calibration and it is obtained to be only 157.4 fg/mL which is sufficiently small for identifying Lep deficiency in the human body. Additionally, the sensor is highly effective for identifying obese conditions under experimental setup.

To check the efficacy of the sensor for detecting Lep in an integrated three-electrode system, we also tested it using SPCE electrode instead of GCE. EIS plots obtained for different concentrations of Lep (500 fg/mL to 80 ng/mL) using SPCE indicated that the value of impedance of the sensors increased linearly with Lep concentration however, not that even as observed when GCE was used as the base substrate. The calibration plot shows that the value of R_{c}t is linear up to 50 ng/mL concentration of Lep. The calculated LOD for this SPCE based Lep sensor was obtained to be approximately 184.9 fg/ml, indicating that the sensor functions perfectly for sensing Lep deficiency and obese conditions regardless of the base substrate. Table 1 shows the how the proposed sensor functions compared to the previously reported Lep sensors. From the table it can be seen that the proposed sensor is better than or comparable to the previously reported Lep sensors.

TABLE 1
Comparison of Lep detection by the proposed sensors
with previously reported detection methods.

| Sensor | Methods | Linear Range | LOD | | References |
|---|---|---|---|---|---|
| Fe:Ni(1:1)/C_GCE | EIS | 500 fg/mL-80 ng/mL | 157.4 | fg/mL | This work |
| Fe:Ni(1:1)/C_SPCE | EIS | 500 fg/mL-50 ng/mL | 184.9 | fg/mL | This work |
| Au/Ce₃NbO₇/CeO₂/ | DPV | 0.5 pg/mL-12 ng/mL | 0.138 | pg/mL | (Liu et al., |
| SPE | | | | | 2021) |
| BSA/anti- | DPV | 0.15 pg/mL-2.5 ng/mL | 0.036 | pg/mL | (Cai et al., |
| leptin/Glu/Cys/ | | | | | 2019) |
| AuNPs/PG-BP/GCE | | | | | |
| — | ELISA | 0.78 pg/mL-50 pg/mL | 0.78 | | (Imagawa |
| | | | | | et al., 1998) |
| rGO-Au-ZZ-BNC | DPV | 1.0 fg/mL-1 ng/mL | 0.87 | fg/mL | (Zhang et |
| | | | | | al., 2022) |
| Fe₃O₄/PDA/Au | Chemiluminescence | 1 pg/mL-800 pg/mL | 0.3 | pg/mL | (He et al., |
| | | | | | 2015) |
| GWEs/PP/MIPL | EIS and | 1 ng/mL-32 ng/mL | 0.11 | ng/mL | (Mihailescu |
| | Conductance | | | | et al., 2020) |
| Apt/Au | EIS | 1.0 pg/mL-1 ng/mL | 0.312 | pg/mL | (ERKMEN |
| NPs/TiO₂ | | | | | et al., 2022) |
| NPs/SPE | | | | | |
| GP electrode | EIS and CV | 0.02 pg/mL-20 pg/mL | 0.00813 | pg/mL | (Özcan and |
| | | | | | Sezgintürk, |
| | | | | | 2021) |

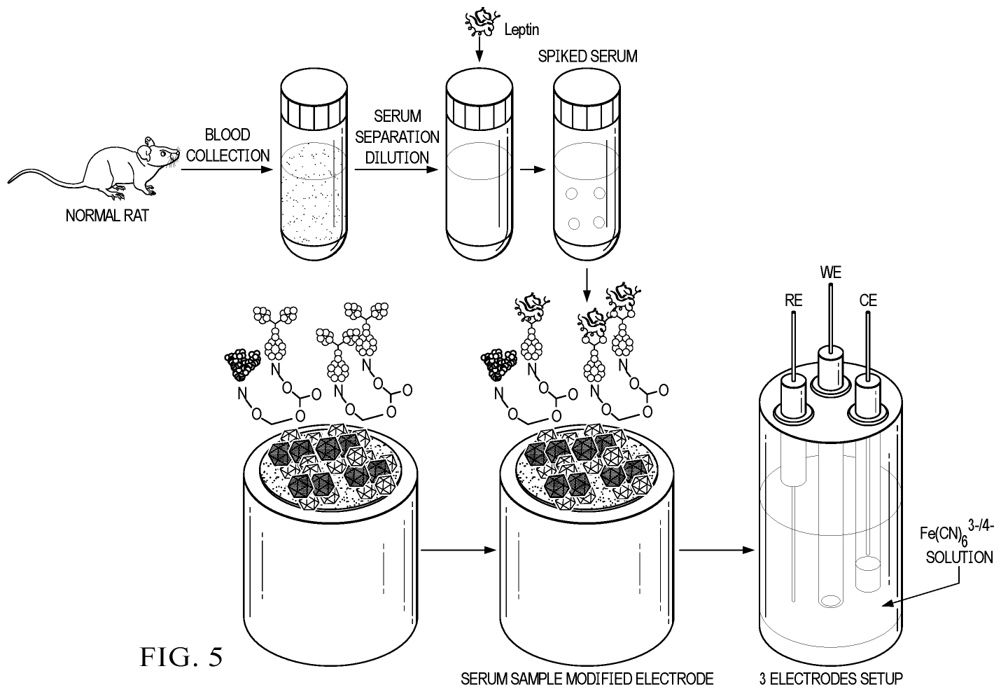

FIG. 5 depicts a diagram illustrating sample collection and electrochemical detection using GCE in accordance with an illustrative embodiment.

For serum analysis, two groups of rats (six HFD rats and six NFD rats) were considered. The body weight and body length of rats increased dramatically after the 15 days period. Once the obese model was created, the animals were euthanized and the blood samples were collected and the serum was separated. Before the analysis, the serum samples of the NFD rats were diluted 100 times and spiked with two different concentrations of Lep (500 pg/mL and 1 ng/mL). All electrodes resulted in comparable R_{ct} values for both concentrations. The percentage of R_{ct} vs number of the serum sample plots demonstrated that the standard deviation for each of the electrode is within 5%. This finding indicates that the sensor is efficient and capable for sensitive detection of Lep from biological sample.

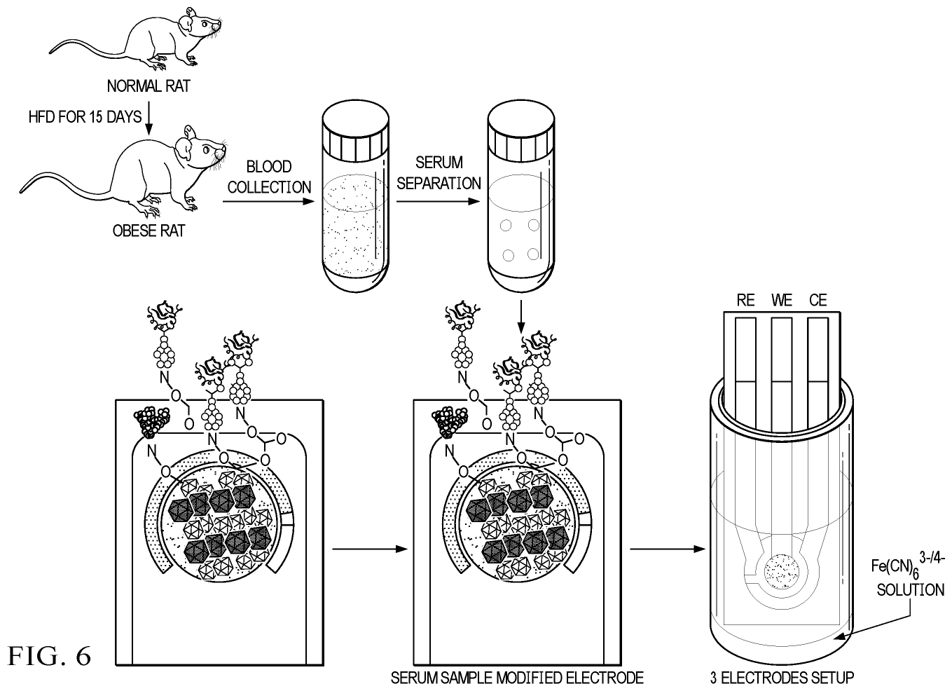

FIG. 6 depicts a diagram illustrating sample collection and electrochemical detection using SPCE in accordance with an illustrative embodiment. Next, the whole serum samples collected from HFD rats were utilized to construct the modified SPCE electrode. The normal level of Lep obtained from HC (healthy control, NFD rats) serum was within the range of 2.3 ng/mL, which is comparable with the previously reported Lep level in normal conditions. The HFD rat serum analysis by the proposed sensor resulted in a significant increment of the Lep level in FRs (˜9.57 ng/mL) and MRs (˜14 ng/mL), which confirms the sensor's utility and effectiveness for real-time detection of Lep for diagnosis leptin resistance.

The electrochemical biosensors need to be able to selectively identify the target analyte in the presence of interfering species. That is why we have tested the selectivity of the Lep/BSA/anti-lep/EDC-NHS/Fe:Ni(1:1)/C_GCEs in with the following compounds: cTnI₃, VCAM1, IL6, glucose, and BSA. Selectivity test was carried out by modifying each electrode with Lep (100 pg/mL), cTnI₃ (5 ng/mL), VCAM1 (5 ng/mL), IL6 (5 ng/mL), glucose (0.02 g/mL), and 5% BSA. The interfering species concentration was taken to be at least 50 times higher than Lep concentration. The R_{c}t value for Lep modified electrode was much higher compared to the cTnI₃, VCAM1, IL6, glucose, and BSA electrodes. This experiment was repeated three times to ensure the selectivity of the Lep/BSA/anti-Lep/EDC-NHS/Fe:Ni(1:1)/C_GCEs.

The biological samples are complex systems where different chemicals are present together with Lep. We performed the anti-interference test by taking Lep with different interfering species in the same solution. The same concentrations as selectivity test were used for anti-interference test. The results show that the R_{c}t value did not change much when Lep was taken with different interfering species. The ant-interference test was also repeated for three times.

The reproducibility test for the Lep/BSA/anti-Lep/EDC-NHS/Fe:Ni(1:1)/C_SPCE was done by modifying six different GCEs following similar conditions. The results show that the biosensor fabrication process is highly reproducible.

The experimental stability and storage stability of the Lep/BSA/anti-lep/EDC-NHS/Fe:Ni(1:1)/C_SPCE was also tested. 30 EIS experiments were done using the same modified biosensor for determining its experimental stability. The Lep biosensor had excellent experimental stability even after 30 EIS runs. The storage stability was tested for 9 days. The Lep/BSA/anti-Lep/EDC-NHS/Fe:Ni(1:1)/C_SPCE retained almost 89.5% performance on Day 9 compared to Day 1. This result shows good storage stability of the Lep biosensors.

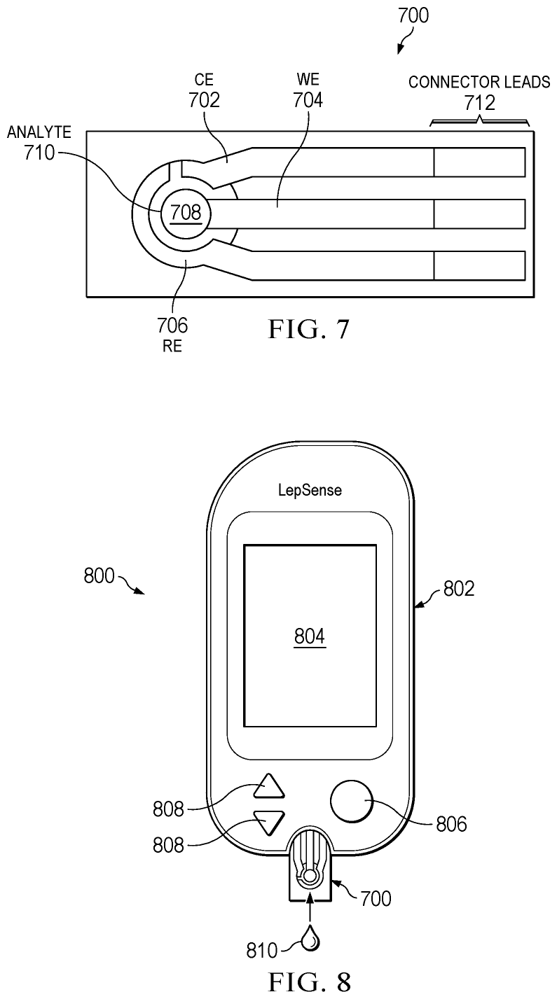

FIG. 7 depicts a diagram illustrating a leptin sensitive electrode in accordance with an illustrative embodiment. Sensor electrode 700 is a screen printed carbon electrode (SPCE) similar to the electrode used in the testing setup shown in FIG. 6.

Sensor electrode 700 comprises a counter electrode (CE) 702, a working electrode (WE) 704, and a reference electrode (RE) 706. WE 704 has an electroactive surface area 708 on which an analyte 710 (e.g., blood sample) may be placed for analysis.

Sensor electrode 700 has a Fe:Ni/C layer deposited on CE 702, WE 704, and RE 706. The Fe:Ni/C layer may comprise Fe:Ni(1:1)/C, Fe:Ni(1:3)/C, or Fe:Ni(3:1)/C. Depositing a biological tissue sample (e.g., blood) containing leptin above a threshold level onto the Fe:Ni/C layer produces an electric current through CE 702, WE 704, and RE 706. The threshold level may be set according to the expected normal range of leptin in the blood (0.5-15.2 ng/ml for males, 0.5-12.5 ng/ml for females). Electrode calibration may be achieved using Sandwich Enzyme-Linked Immunosorbent Assay (ELISA). When sensor electrode 700 is connected to a biosensor device (see FIG. 8), the current through CE 702, WE 704, and RE 706 flows to connector leads 712 in the device.

FIG. 8 depicts a diagram illustrating a biosensor device configured to detect leptin in accordance with an illustrative embodiment. Biosensor device 800 comprises a housing 802 into which sensor electrode 700 may be inserted.

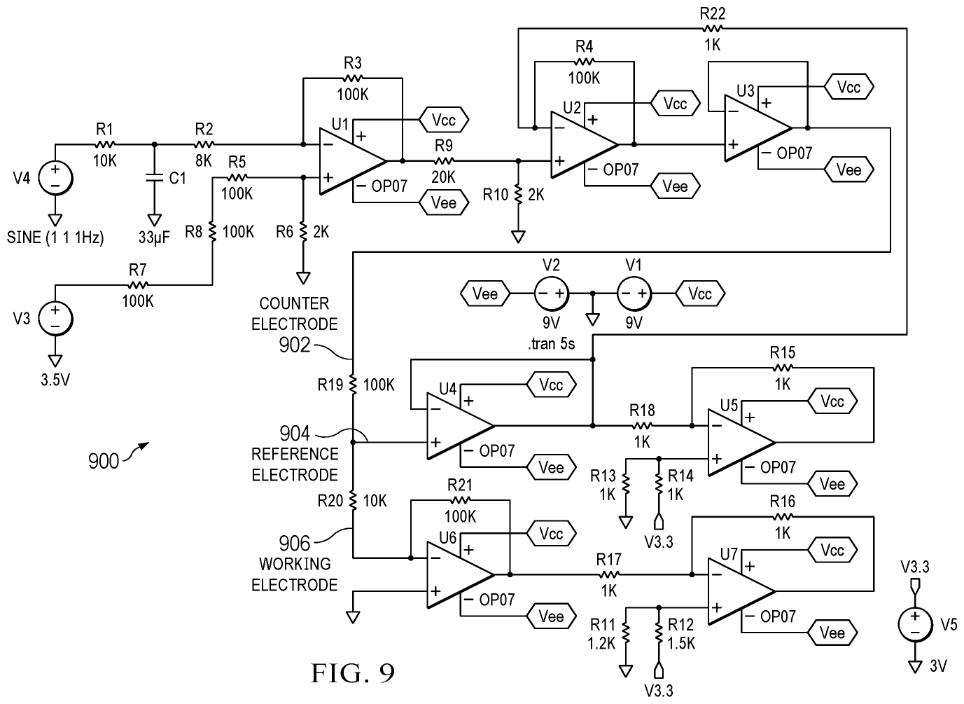

As explained above, when a biological tissue sample 810 (e.g., blood) is placed on sensor electrode 700, an electric current is generated if the biological tissue sample contains leptin above a threshold level. This current is processed by a circuit inside housing 802 (see FIG. 9) connected to CE 702, WE 704, and RE 706. The circuit is configured to produce a specified waveform in response to the current produced by the presence of leptin above the threshold level.

The waveform produces a readout which may be displayed on display 804. Biosensor device 800 can be turned on and off via button 806. Buttons 808 may be used to scroll through data on display 804.

FIG. 9 depicts a circuit diagram of a leptin level processing circuit in accordance with an illustrative embodiment. Circuit 900 depicts the complete circuit inside the biosensor with the sensor electrode 700 inserted as shown in FIG. 8. FIG. 9 shows the counter electrode 902, reference electrode 904, and working electrode 906 relative to the rest of the leptin processing circuit 900.

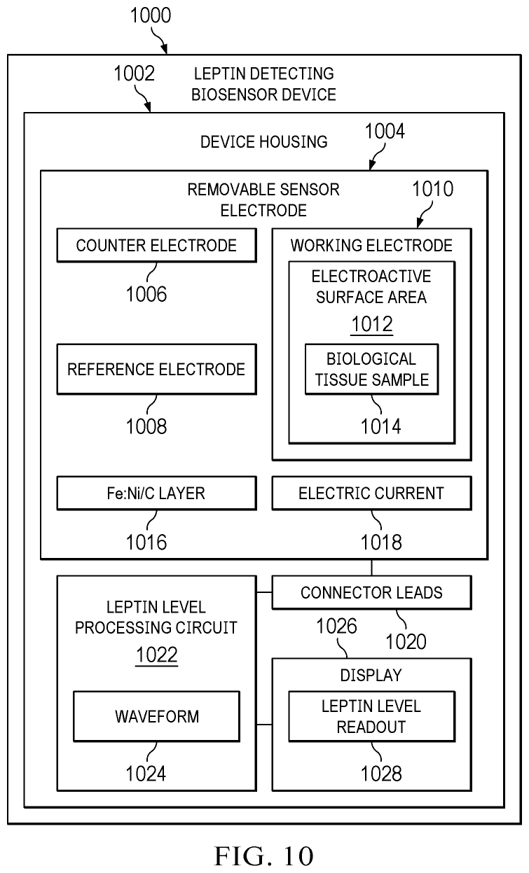

FIG. 10 depicts a block diagram illustrating a leptin detecting biosensor device in accordance with an illustrative embodiment. Biosensor device 1000 may be an example implementation of leptin processing circuit 900 in FIG. 9.

Biosensor device 1000 comprises a housing 1002 that contains the components of the device. Housing 1002 may be a hand-portable/handheld housing analogous to a glucometer.

Removable sensor electrode 1004 can be inserted and removed from housing 1002. Sensor electrode comprises a counter electrode 1006, a reference electrode 1008, and a working electrode 1010. Working electrode 1010 includes an electroactive surface area on which a biological tissue sample 1014 may be placed.

At least a portion of the removable sensor electrode 1004 is covered with a Fe:Ni/C layer 1016. This layer may comprise Fe:Ni(1:1)/C, Fe:Ni(1:3)/C, or Fe:Ni(3:1)/C. If the biological tissue sample contains a concentration of leptin above a defined threshold, the removable sensor electrode 1004 generates an electric current 1018 that is proportional to the concentration of leptin in the biological tissue sample.

When removable sensor electrode 1004 is inserted into housing 1002 it is in electrical contact with leptin level processing circuit 1022 via connector leads 1020. The counter electrode 1006, reference electrode 1008, and working electrode 1010 are in electrical contact with respective connector leads 1020. Connector leads 1020 carry the electric current 1018 to leptin level processing circuit 1022, which produces a waveform 1024 in response to the current. Leptin level processing circuit determines the level of leptin in the biological tissue sample 1014. The level is then displayed as a leptin level readout 1028 on display 1026.

As used herein, the phrase “a number” means one or more. The phrase “at least one of”, when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

For example, without limitation, “at least one of item A, item B, or item C” may include item A, item A and item B, or item C. This example also may include item A, item B, and item C or item B and item C. Of course, any combinations of these items may be present. In some illustrative examples, “at least one of” may be, for example, without limitation, two of item A; one of item B; and ten of item C; four of item B and seven of item C; or other suitable combinations.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different features as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. A leptin detecting device, comprising:
a counter electrode;
a reference electrode;
a working electrode having an electroactive surface area;
a Fe:Ni/C layer deposited on the counter electrode, reference electrode, and working electrode, wherein deposit of a biological tissue sample containing a concentration of leptin above a threshold level onto the Fe:Ni/C layer produces an electric current through the counter electrode, reference electrode, and working electrode; and
a circuit connected to the counter electrode, reference electrode, and working electrode, wherein the circuit is configured to produce a specified waveform in response to the electric current produced by the presence of leptin above the threshold level.

2. The device of claim 1, wherein the device comprises a screen printed carbon electrode device.

3. The device of claim 1, wherein the device comprises a glassy carbon electrode device.

4. The device of claim 1, wherein the Fe:Ni/C layer comprises one of:
Fe:Ni(1:1)/C;
Fe:Ni(1:3)/C; or
Fe:Ni(3:1)/C.

5. The device of claim 1, further comprising a display that displays a leptin level readout in the biological tissue sample.

6. The device of claim 1, wherein the strength of the electric current is proportional to the concentration of leptin in the biological tissue sample.

7. The device of claim 1, wherein the circuit is contained in a hand-portable housing, and wherein the counter electrode, reference electrode, and working electrode together comprise a removable sensor electrode that can be inserted and removed from the hand-portable housing.

8. The device of claim 7, wherein the counter electrode, reference electrode, and working electrode contact connector leads in the housing that carry the electric current to the circuit.

9. A biosensor, comprising:
a housing;
a removable sensor electrode inserted into the housing, wherein the sensor electrode comprises a Fe:Ni/C layer, wherein deposit of a biological tissue sample containing a concentration of leptin above a threshold level onto the Fe:Ni/C layer produces an electric current through the sensor electrode; and
a circuit inside the housing, wherein the circuit is electrically connected to the sensor electrode, and wherein the circuit is configured to produce a specified waveform in response to the electric current produced by the presence of leptin above the threshold level.

10. The biosensor of claim 9, wherein the sensor electrode comprises a screen printed carbon electrode device.

11. The biosensor of claim 9, wherein the sensor electrode comprises a glassy carbon electrode device.

12. The biosensor of claim 9, wherein the Fe:Ni/C layer comprises one of:
Fe:Ni (1:1)/C;
Fe:Ni(1:3)/C; or
Fe:Ni(3:1)/C.

13. The biosensor of claim 9, further comprising a display that displays a leptin level readout in the biological tissue sample.

14. The biosensor of claim 9, wherein the strength of the electric current is proportional to the concentration of leptin in the biological tissue sample.

15. The biosensor of claim 9, wherein the housing is hand-portable.

16. The biosensor of claim 9, wherein the removable sensor electrode contacts connector leads in the housing that carry the electric current to the circuit.

17. A leptin biosensor, comprising:
a handheld housing;
a screen printed carbon electrode (SPCE) removably inserted into the handheld housing, the SPCE comprising:
a counter electrode;
a reference electrode;
a working electrode;
a Fe:Ni/C layer deposited on the SPCE, wherein deposit of a blood sample on the Fe:Ni/C layer produces an electric current through the SPCE if the blood sample contains a concentration of leptin above a threshold level;
a circuit electrically connected to the SPCE, wherein the circuit is configured to produce a specified waveform in response to the electric current produced by the presence of leptin above the threshold level; and
a display in the housing that displays a readout of detected lepton levels in response to the waveform produced by the circuit.

18. The leptin biosensor of claim 17, wherein the Fe:Ni/C layer comprises one of:
Fe:Ni(1:1)/C;
Fe:Ni(1:3)/C; or
Fe:Ni(3:1)/C.

19. The leptin biosensor of claim 17, wherein the strength of the electric current is proportional to the concentration of leptin in the biological tissue sample.

20. The leptin biosensor of claim 17, wherein SPCE contacts connector leads in the housing that carry the electric current to the circuit.
